(19) United States
(12) Patent Application Publication
(10) Pub. No.: US 2021/0153528 A1
(43) Pub. Date: May 27, 2021

(54) SYSTEM, METHOD AND APPARATUS FOR STERILISATION OF MATERIALS

(71) Applicant: VAK-TEK PTY LTD, South Australia (AU)

(72) Inventors: Brett John YOUNG, South Australia (AU); Joyce DARCY, South Australia (AU); Ivan WINTER, South Australia (AU); Simon Patrick KENNEDY, South Australia (AU)

(21) Appl. No.: 16/618,250

(22) PCT Filed: May 21, 2018
(86) PCT No.: PCT/AU2018/050483

§ 371 Date: Nov. 29, 2019

(30) Foreign Application Priority Data
May 30, 2017 (AU) 2017902042

(51) Int. Cl. A23L 3/015; B65D 88/54

(52) U.S. Cl. CPC A23L 3/0155; A23B 7/148; B65D 88/54

(57) ABSTRACT

An apparatus for sterilising material comprising a first pressure vessel, in which the pressure vessel has a vessel infeed and a vessel outlet. The vessel infeed and vessel outlet are adapted to form respective seals with the pressure vessel such that a desired pressure can be maintained in the pressure vessel, and wherein pressure vessel is depressurised such that the material deposited in the pressure vessel is sterilised and at least a portion of the inner surfaces of the pressure vessel are sterilised, such that the material deposited into the pressure vessel can be stored therein for a predetermined period.

### TECHNICAL FIELD

The present invention relates to a system, method and apparatus for sterilisation of material. More particularly, the present invention may relate to a system, method and apparatus for sterilisation of organic particulates, namely grains and nuts.

### BACKGROUND

Storage of organic materials, particularly organic particulate materials such as rice, wheat, oats, flour, muesli, nuts, berries, fruits, liquids, soups, gruel, cereals, and other organic materials is costly and requires particular care in relation to safety and storing organics materials, especially when considering reducing wastage of stored materials.

Particulate materials, such as grain, are typically stored in silos before being transported, processed and packaged. While being stored in silos the particulate material is often exposed to pests, insects and microbiological contaminants which may destroy or reduce the quality of the particulate material being stored in the silo. Typical silos may often result in large amounts of stored food being lost due to the above exposure to pests, insects and microbiological contaminants, and therefore is a strain on the overall efficiency of production and profits which is particular concern due to an ever increasing global population.

Typically, the particulate materials are only sterilised or cleaned of contaminants during the packaging process and therefore have a significant period of time in which they can be exposed to a number of pests, contaminants and oxygen. This exposure can also reduce the shelf-life of the goods being sold, overall profits and increases wastage of the materials.

Sterilisation and storage of high volumes of materials is difficult, especially when attempting to maintain the integrity of the food being stored. Storage of food stuffs is generally more reliable when the food is sterilised and the storage vessel for the food is also sterilised. However, maintaining sterilisation is difficult for large volumes of food without packaging and sealing food from air or contaminants.

Common food storage means include concrete silos or bunkers are the most common high volume storage options, however they are exposed to atmospheric conditions and also allow for microorganisms to effect the materials stored therein. These silos also allow unwanted rodents and pests to enter into the silos which can further impact the quality of the food, and volumes available for sale. Fermentation is a further problem associated with storing foods, which can also degrade the quality of the food.

Low-oxygen silos are another option designed to keep the contents (foods) of a silo in a low-oxygen atmosphere, to keep fermented contents in a high quality state, and to prevent microorganisms negatively impacting the contents of the silo or bunker. Low-oxygen silos are only opened directly to the atmosphere during the initial loading, and even the unloader chute is sealed against air infiltration, although they are still indirectly exposed to the atmosphere external the silo. During the time that the silos are exposed to the atmosphere, the contents of the silo can also harbour pests, rodents and other microorganisms which may also impact the food. It will be understood that these silos are merely sealed without a change of internal oxygen levels.

The low-oxygen silo is open to the atmosphere but outside air is separated from internal air by large impermeable bags sealed to the silo breather openings. In the warmth of the day when the silo is heated by the sun, the gas trapped inside the silo expands and the bags “breathe out” and collapse. At night the silo cools, the air inside contracts and the bags “breathe in” and expand again. However, due to the low-oxygen silo design these silos typically do not have a suitable output rate compared to that of concrete silos and bunkers. Further, the costs associated with the unloader of the low-oxygen silos is significant such that loss of stored food contents typically outweighed the benefit of the low-oxygen atmosphere in the silo.

Further, these silos do not allow for sterilisation of the contents of the food, nor can they provide a sterilised environment which can reduce potential for spoiling of contents stored in the silo. Sterilisation may occur only after the contents are removed and is costly and time consuming to effectively sterilise the interior of the silo. However, pre-contents sterilisation is typically ineffective due to the contents commonly containing undesirable biological material and potentially pests.

Conventionally, solid ingredients of nourishing preparations containing both solid and liquid such as stew and sold as preservable food are sterilised on a batch basis in sterilising vessels before being retrieved for packaging. There are known sterilising apparatuses for producing sterilised solid food where granular solid food is mixed with liquid serving as a heat transmitting medium, heated, sterilised and then separated again from the liquid. However, the known sterilising vessels have drawbacks that require cumbersome operations of mixing solid with liquid before heating and sterilising the food and pumping out the liquid after the completion of sterilization and that the solid food is liable to be broken as the container of the apparatus is rotated while the food is heated for sterilization.

In addition, medium- to long-term food storage devices also present well known problems, for example; contamination, oxidation, desiccation, bacterial and fungal decay, and infestation by pests such as insects and mites, and also by rodents. The percentage loss of stored food can be significantly high despite the use of current sterilisation methods, and on a world scale can amount to up to around one third of stored food can be lost.

Contemporary sterilisation or disinfestation methods include chemical fumigation, ionising radiation, and conventional hot air treatment, and dielectric heating, that is radio frequency microwave energy. It is estimated that more than twenty thousand species of pests destroy about one third of the worlds' food production annually. Current technology is to fumigate grain with substances such as phosphine however this may potentially be carcinogenic, and pests, bacteria, fungi, and mites are able to breed up a resistance, which is a general problem of current sterilisation and disinfectant methods and devices. It is therefore advantageous to provide for a storage vessel or device to overcome at least one known issue of the prior art.

Any discussion of the prior art throughout the specification should in no way be considered as an admission that such prior art is widely known or forms part of common general knowledge in the field.

### SUMMARY

### Problems to be Solved

It may be advantageous to provide a storage means for organic material which may be used to effectively sterilise the contents of the storage means.

It may be advantageous to provide a storage means for organic material which may allow a low-oxygen environment to be maintained.

It may be advantageous to provide a storage vessel for materials which may be efficiently cycled to another storage vessel.

It may be advantageous to provide a storage vessel for materials which may be sterilised with organic material stored in the storage vessel.

It may be advantageous to provide a storage vessel for materials which may be maintained at a desired temperature.

It may be advantageous to provide a storage vessel for materials which may be maintained at a desired pressure.

It may be advantageous to provide a storage vessel for materials which may be maintained at a desired oxygen level.

It may be advantageous to provide a storage vessel for materials which may effectively remove microbiological organisms from the storage vessel.

It may be advantageous to provide a storage vessel for materials which may reduce the potential for explosions or fires.

It may be beneficial to provide an apparatus which reduces the rate of fermentation of stored food materials.

It is an object of the present invention to overcome or ameliorate at least one of the disadvantages of the prior art, or to provide a useful alternative.

### Means for Solving the Problem

An aspect of the present invention may relate to an apparatus for sterilising material, the apparatus comprising; a first pressure vessel; the pressure vessel may comprise a vessel infeed and a vessel outlet. The vessel infeed and vessel outlet may be adapted to form respective seals with the pressure vessel such that a desired pressure can be maintained in the pressure vessel. The pressure vessel may be deprived of at least portion of atmosphere such that the material deposited in the pressure vessel may be sterilised and at least a portion of the inner surfaces of the pressure vessel are sterilised, such that the material deposited into the pressure vessel can be stored therein for a predetermined period of time.

In at least one embodiment, the pressure vessel maybe a plurality of pressure vessels, in which at least a first pressure vessel and a second pressure vessel of the plurality of pressure vessels are in communication with each other. Preferably, when the first pressure vessel may be depressurised, the second pressure vessel may be partially depressurised by opening a fluid conduit between the first pressure vessel and the second pressure vessel. Preferably, the material deposited into a first pressure vessel can be cycled to a second pressure vessel to be stored. Preferably, a sterilant gas may be injected into the pressure vessel a predetermined pressure. Preferably, the gas may be an inert gas at a predetermined pressure. Preferably, the desired pressure and the predetermined pressure may be the same pressure. Preferably, an infeed hopper may be disposed above the pressure vessel and adapted to direct material to the inlet of the pressure vessel. Preferably, an outlet hopper may be disposed relatively below the pressure vessel and adapted to receive material from the outlet of the pressure vessel. Preferably, the pressure vessel may be a vertical pressure vessel such that material can be gravity fed into the pressure vessel. Preferably, the pressure in the vessel may be adapted to cycle between one atmosphere to the desired pressure. Preferably, the pressure vessel may be cycled three times between one atmosphere to the desired vacuum pressure. Preferably, the three cycles may occur during a period in the range of 15 minutes to 45 minutes.

Another aspect of the present invention may relate to a method of sterilising material in a multi-pressure vessel apparatus, the method may comprise the steps of; depositing a material in a first pressure vessel; sealing the first pressure vessel and creating a depressurised atmosphere in the first pressure vessel; maintaining the depressurised atmosphere in the pressure vessel for a first predetermined holding time; depositing a material into a second pressure vessel; sealing the second pressure vessel and opening a fluid conduit between the first pressure vessel and the second pressure vessel such that the depressurised atmosphere from the first pressure vessel is used to partially depressurise the atmosphere of the second pressure vessel; closing the fluid conduit between the first pressure vessel and the second pressure vessel; and creating the depressurised atmosphere in the second vessel for a second predetermined holding time.

In at least one embodiment, the method preferably further may comprise the step of injecting a gas into the first pressure vessel and/or the second pressure vessel during the respective holding time. The method may preferably further comprise the step of opening the seal of the first pressure vessel such that material deposited in the first pressure vessel can be extracted via gravity feed. Preferably, the method further comprises the step of cycling the pressure in at least one of the first vessel and the second vessel from between one atmosphere and the depressurised atmosphere.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates a perspective view of an embodiment of the apparatus of the present disclosure for sterilising and storing a material;

FIG. 2 illustrates a side view of embodiment of a food sterilisation apparatus with a plurality of pressure vessels;

FIG. 3 illustrates a top perspective view of an embodiment of a sterilisation apparatus with transparent view of an infeed;

FIG. 4 illustrates a side view of the top of an embodiment of an sterilisation apparatus with multiple pressure vessels;

FIG. 5 illustrates a side view of the bottom of an embodiment of an sterilisation apparatus with multiple pressure vessels;

FIG. 6 illustrates a top view of an embodiment of the apparatus of the present invention in which the pressure vessels are evenly spaced; and

FIG. 7 illustrated yet a further embodiment of an apparatus comprising a flexible pressure vessel and frame structure.

### DESCRIPTION OF THE INVENTION

Preferred embodiments of the invention will now be described with reference to the accompanying drawings and non-limiting examples.

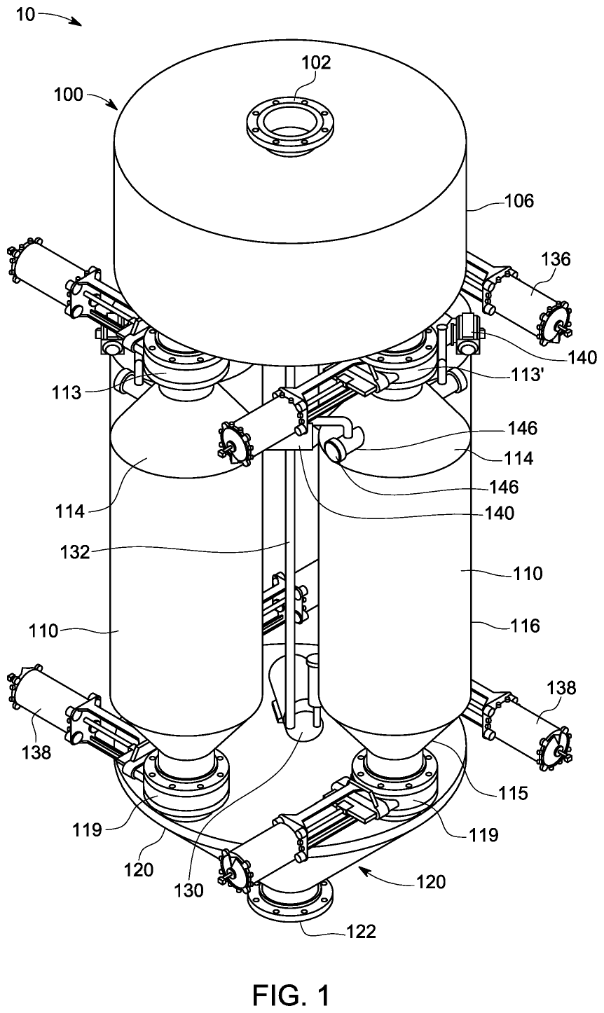

Referring to FIG. 1 there is depicted an embodiment of a multi-pressure vessel apparatus 10, herein referred to as apparatus 10, for storing organic material. The pressure vessels 110 may be at least one of a silo, a silage or a bunker which can be used to store a particulate material, such as rice, wheat, oats, flour, muesli, nuts, berries, fruits, liquids, soups, gruel, cereals, and other organic materials or foods. The apparatus 10 is preferable used to contain organic material which can be sterilised or otherwise be removed of a majority of biological contaminants or microorganisms which may adversely impact the organic material to be stored. Typically microorganisms may promote decay or growth of mould which can reduce the usable amount of organic material being stored. This results in wastage of the organic material and the overall amount of money that the organic material can be sold for. Therefore, there is a need to provide a means for storing organic food materials which reduces the potential for biological contaminants, microorganisms and oxygen to be negatively impacted.

The pressure vessels 110 may also be referred to as vacuum vessels or silos, however unlike known silos, the vessels may be depressurised or have an atmosphere altered after sealing of the vessel/silo. Material, such as grain, nuts, legumes, particulate, or more generally food, can be fed into the apparatus 10 via an infeed hopper 100 or infeed 100 at the top of the apparatus 10. The infeed hopper 100 may have an inlet 102 and an inlet seal (not shown) or inlet hatch (not shown) which can seal or block the inlet 102 of the infeed hopper 100 such that new materials cannot enter the infeed hopper 100. The infeed hopper 100 further comprises at least one wall 106, such as a circular wall 106. The food may be conveyed to the infeed via a pneumatic conveyor, a bucket elevator or any other known method of transporting material to the infeed hopper 100. To introduce the food into at least one of the vessels 110, the infeed valve 113 of the infeed 112 of the first vessel 110A may be open such that the food, typically grain, flour or another particulate can enter into the vessel 110. The infeed valve may be operated via an infeed sealing means 136 which may be an electronic means (such as a solenoid) or manually operated by a technician or user of the apparatus 10. The infeed 112 is preferably adapted to close, via infeed valve 113, when the respective vessel 110 is at capacity or “full”. Sensors (not shown) may be provided to determine a remaining capacity of a vessel. Allowing the infeed 112 to open and close when the vessel 110 is at capacity provides the advantage that the free gas (typically air) within the vessel is minimised as the majority of the volume of the vessel 110 is occupied by the food. During loading the outlet 122 is closed, preferably via hopper outlet seal (not shown), such that the food is retained in the vessel 110. Optionally, instead of a hopper outlet seal, a sealing means 138 is used to seal and retain the material loaded into at least one vessel 110. An infeed valve 113 is preferably provided at the infeed 112 to form a seal to at least substantially retain an internal pressure or atmosphere of the pressure vessel 110.

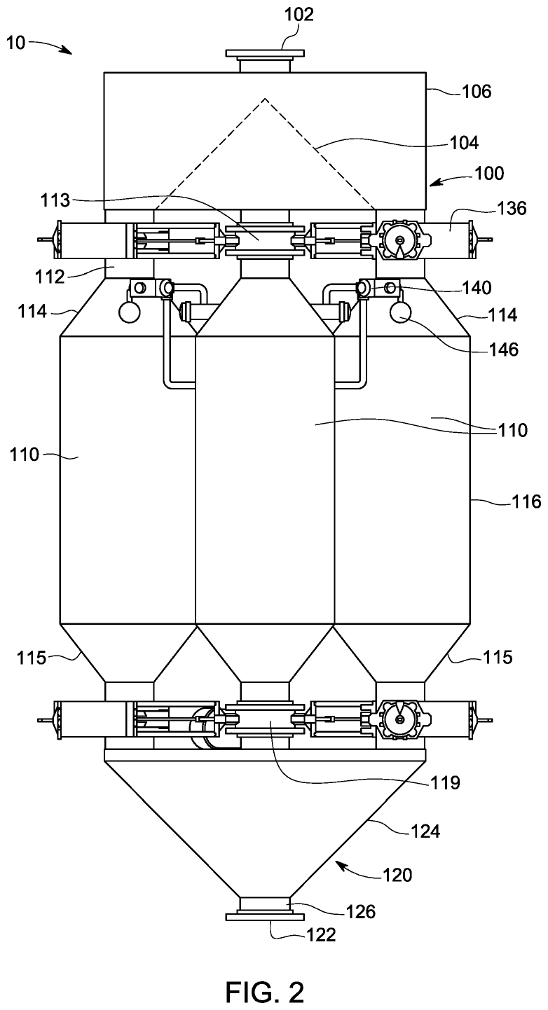

Referring to FIG. 2, there is an embodiment of an apparatus 10 comprising a plurality of pressure vessels 110 with an infeed hopper 100 comprising a feed diverter 104 which is internal to the infeed hopper 100. The infeed diverter 104 is used to direct materials fed into the infeed hopper 100 to a vessel infeed 112 and is shown as a conical shape, although any shape may be used such as a ‘reamer’ shape (similar in appearance to that of the juicer portion of a hand citrus juicer). If a reamer shape is used respective depressions in the reamer may be disposed facing a respective vessel infeed 112. Each vessel infeed 112 is preferably fitted with an infeed valve 113. The infeed valve 113 may be operated remotely and an electronic manipulation means may be used to engage and disengage the infeed valve 113. In one embodiment, the manipulation means 136 may be a solenoid. It will be appreciated that a manual infeed valve (not shown) may be used wherein a user is required to seal the infeed manually 112, for example via a wheel valve.

Each pressure vessel 110 may all be of a similar shape, volume and dimensions to allow for storing of materials so that the apparatus 10 is not off balance by loading larger vessels 110. It will be appreciated that while it is preferred that each vessel 110 is of a similar dimensions, volume, and/or shape, the vessels 110 may have any desired dimensions, volume, and/or shape. The top shape 114 of the pressure vessel 110 and the bottom shape 115 of the vessel 110 are preferably conical, which is typically advantageous as this can limit the flow into and out of the vessel 110. The walls 116 of the vessel 110 extend between the larger annulus of each of the top shape 114 and the bottom shape 115. Preferably the walls 116 form a cylinder which allows for even distribution of forces radially and provides structurally stability when a hydrostatic force is applied to the walls 116. The walls 116 may collectively refer to all the walls of the vessel which includes the top shape 114 and the bottom shape 115.

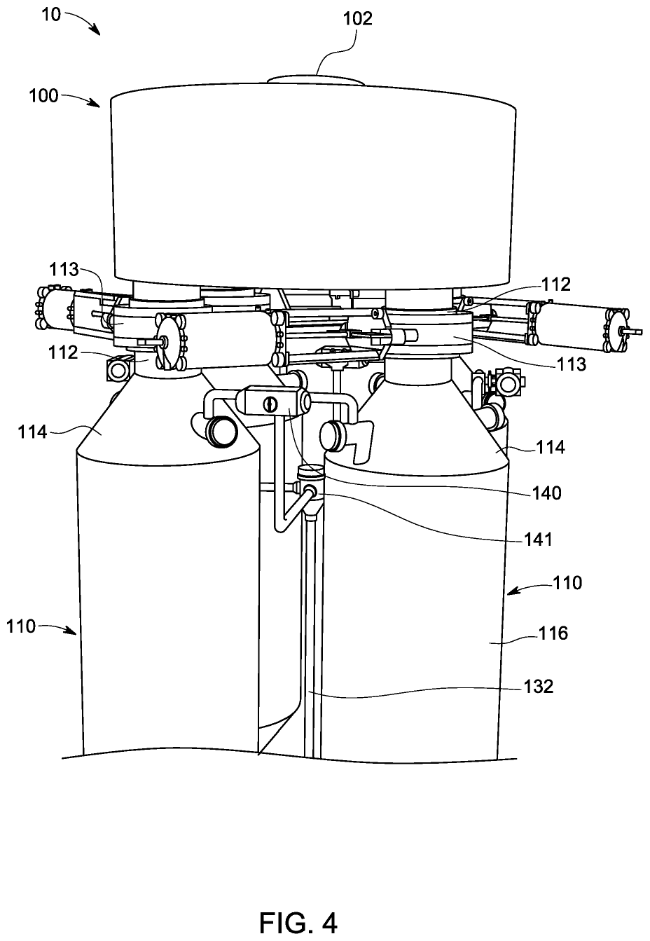

After loading the vessel 110, the infeed 112 can be closed and the gas (a gas may more broadly be a fluid which includes gas and/or liquid) in the vessel 110 can be at least partially removed via a vacuum pump 130. The pump 130 may be fitted with a filtration means, such as a filter portion 134. When the vacuum pump 130 is activated, a vacuum may be created which extends to a distribution manifold 140 (see FIG. 4) such that the distribution manifold 140 generates a vacuum at vacuum valve 142, and if the vacuum valve 142 is open the vacuum draw atmosphere from the vessel 110. The pump 130 and the manifold 140 are connected via a pump conduit 132. The pump conduit 132 may be connected to a manifold hub 141 which connects to a plurality of manifolds 140. The manifolds 140 may broadly be referred to as a fluid conduit or fluid conduit valve which allows for selected passage of fluids, preferably between vessels 110, the pump 130 and/or the exterior of the apparatus 10. While there may be more than one manifold 140, for example a first manifold 140 between the first vessel 110A and second vessel 110B, and a second manifold 140 between the second vessel 110B and a third vessel 110C, throughout this specification they may be collectively referred to as a manifold 140. Preferably, each vessel 110 comprises a respective vacuum valve 142 such that the respective valve may be used to create a vacuum in a respective vessel 110. Preferably, each vessel 110 of the apparatus is evenly spaced with respect to a central vertical axis of the apparatus 10.

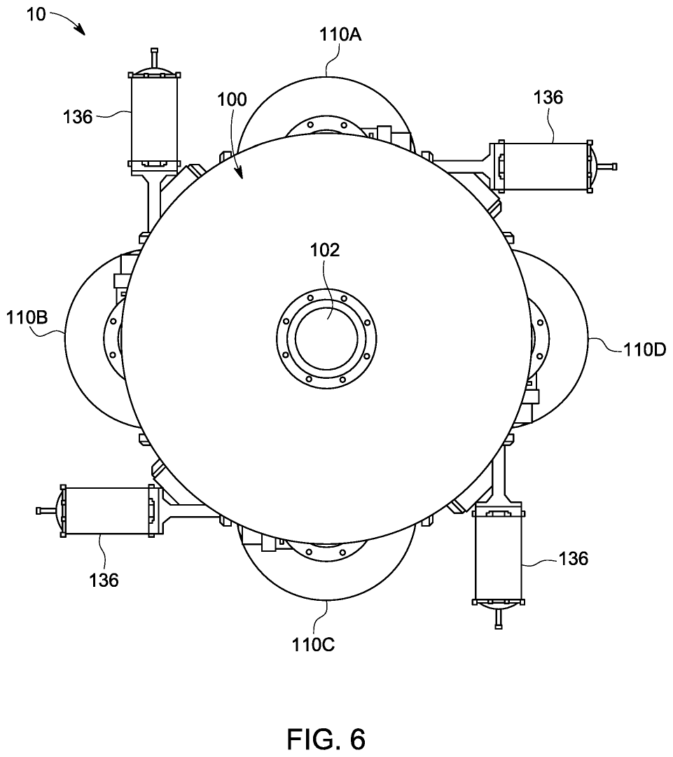

Once the vacuum is generated at the vacuum valve 142, at least one vacuum valve 142 of the manifold 140 can be opened such that gas and/or fluid is drawn out of the vessel 110. It will be appreciated that only a portion of the gas may be withdrawn from the vessel 110. If only one vessel vacuum valve 142 was opened, a subsequent or desired vacuum valve 142A may be opened to withdraw gas from the next respective subsequent or desired vessel. For example, and with reference to FIG. 6, if the vacuum valve 142A of the first vessel 110A is opened to draw out gas, a second vessel 110B may have its vacuum valve 142B opened to withdraw the gas from said second vessel 110B. It will be appreciated that the first vacuum valve 142A may or may not be closed. If the first vacuum valve 142A is not closed, the vacuum within the first vessel 110A may be used to create a partial vacuum in the second vessel 110B as the pressure between the first vessel 110A and the second vessel 110B reaches equilibrium. If the first vessel 110A is used to generate a vacuum in a second vessel 110B, the first vessel 110A has preferably completed a sterilisation process, however this is optional.

The route for the gas drawn from the vacuum will comprise at least one filter 144 or other means for limiting or restricting drawing of particulate material, dust or powder from the vessel 110. Preferably, the at least one filter is a vacuum valve filter 144 positioned proximal the vacuum valve 142. A further outlet filter 146 may be provided to restrict particulate material, dust or powder from being removed from the vessel 110 into the atmosphere external the apparatus 10. The vacuum in the vessel 110 may be drawn to around twenty-nine (29) inches of mercury (approximately 100 kPa). It will be appreciated that the vacuum in the vessel 110 may be in the range of around 5% to 100%, or preferably 25% to 90%, or more preferably, 50% to 85%. In one exemplary embodiment, the vacuum may be 75% to 85%. Further, the vacuum may be in the range of between 10 kPa to 150 kPa, or preferably, 40 kPa to 120 kPa, or more preferably between 50 kPa to 110 kPa, even more preferably, the range is 55 kPa to 101 kPa. The vacuum may be held in the vessel for a predetermined period of time, for example a time of around two (2) minutes may be sufficient to kill most forms of undesirable microorganisms in the vessel. However, while two (2) minutes is exemplified, this time may be any predetermined period of time. Any combination of vacuum and time may be used and the apparatus 10 is not limited to the above exemplified ranges, times or vacuum percentages. The period of time in with the vacuum is generated within the vessel 110 may be referred to as a ‘dwell time’. Preferably, the internal temperature of the vessel 110 is reduced when the vacuum is imparted to the vessel(s) 110. Further, vacuuming the vessels 110 may assist with drying of contents, and as such cycling vacuum pressures may be used to assist with drying contents in the vessel, which can further save foods stored from adverse effects such as saturation or rotting.

It may be a further advantage to cycle pressure as the act of cycling pressure increases apotheosis of living organic material. Further, cycling may reduce the potential for spores to survive the depressurisation step.

If the apparatus 10 is adapted to vacuum only one vessel at a time, after the first vessel 110 dwell time is complete the vacuum valve for each of the first and the second vessels 110A, 110B may be opened. Opening both first vessel and the second vessel inlet valves for is used to equalise the vacuum within the first and the second vessels instead of vacuuming the second vessel from atmospheric pressure, which will save energy and reduce operational costs. This provides a significant advantage over known silos, bunkers and pressure vessels. During the vacuum equalisation between the first vessel and the second vessel will preferably be via a path with filters to prevent dust, powder or contaminants from being transferred between the vessels 110 or into the manifold 140. The vacuum valve 142A for the first vessel is closed once equilibrium of the internal pressures between the first and second vessels, 110A, 110B is obtained, then the vacuum pump extracts atmosphere from the second vessel 110B to the desired vacuum to be maintained for the desired dwell time. The dwell time for the second vessel may not be the same as the dwell time for the first vessel as the partial vacuum generated by opening the inlets to both the first and the second vessels may alter the required dwell time. It will be appreciated that at least a partial vacuum may be maintained after the dwell time within the vessel(s), which may reduce internal temperatures and reduce the oxygen content. Reducing internal temperatures and reducing the oxygen content will generally improve the safety of the apparatus 10 as this will reduce the potential for fermentation, fires and/or explosions to occur within the vessel(s). Therefore, the present invention may provide for a more cost effective and safer apparatus 10 to store food materials compared to known silos, granaries and bunkers.

Optionally, a supplemental fluid may be injected into a vessel 110 after a dwell time which may be used to sterilise the interior of the vessel 110 from contaminants. The supplemental fluid may be used to increase certainty that the interior of the vessel 110 is sterilised. Preferably, the fluid is a gas which does not impact the quality of the food being stored, and preferably does not leave a chemical residue. For example, the gas may be ozone, Ethylene oxide, a nitrous gas, sulphite gas, sulphur dioxide, a gas with a high percentage of nitrogen or any other gas which may be used to kill or neutralise microorganisms within a pressure vessel 110 without adversely impacting the food contents. Within the localised conditions of the vessel 110 the gas may be effectively inert or generally non-reactive with the contents of the vessel 110. Optionally, the fluid may be used prior to the food being deposited within the vessel 110 such that the interior of the vessel 110 can be sterilised.

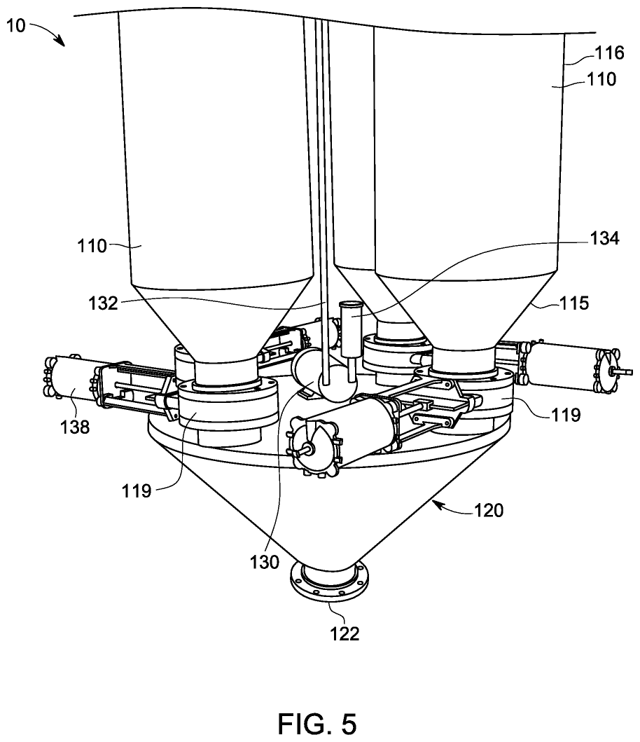

After the sterilisation of the food, the food may be stored in the apparatus 10 or may be extracted for transportation or packaging. Referring to FIG. 5, when the food is desired to be extracted from the vessel 110, the vessel outlet valve 119 of the vessel outlet 118 is opened to allow the vessel to be released from the vacuum (if present) and allow movement of the materials within the vessel 110. Once the vessel outlet valve 119 is opened the food may be allowed to flow from the vessel 110 through the outlet hopper 120 to be stored elsewhere, transported or used in any other desired manner. After the vessel 110 is emptied, the vessel outlet valve 119 may close and the interior of the vessel 110 may be optionally cleaned. The vessel outlet valve 119 may be actuated via a solenoid or other suitable sealing means 138 for closing and opening the vessel outlet 118. It will be appreciated that each vessel 110 may be pressurised, vacuumed, filled, stored or emptied individually without impacting the environments of the other vessels 110 of the apparatus 10. In one embodiment, the vacuum valve 142 may be a three-way (three port) vacuum valve, a two way (two port) valve, or a four way (four port) valve. The vacuum valve 142 may be any one of; a hydraulic valve, a pneumatic valve, a manual valve, a solenoid valve or any other desired valve. At least one of the inlets, valves and/or other moving components of the apparatus 10 may be operated via a controller (not shown). The controller may be in communication with with a computer system which may be remotely accessed such that the apparatus 10 can be controlled at a remote location, or operated on site. The apparatus 10 may be adapted to be controlled via an internet connection, satellite connection, Bluetooth™ and/or Wi-Fi connection.

In yet a further embodiment, the apparatus 10 or components thereof may be controlled via hardware and an operating environment which may include a general purpose computing device (e.g., a personal computer, workstation, mobile device, smart phone, smart device, or server), including one or more processing units, a system memory, and a system bus that operatively couples various system components including the system memory to the processing unit. There may be more than one processing unit, such that the processor of computer comprises a single central-processing unit (CPU), or a plurality of processing units, commonly referred to as a multiprocessor or parallel-processor environment. In various embodiments, computer is a conventional computer, a distributed computer, or any other type of computer.

A number of ancillary devices, systems and/or apparatuses may be used with the apparatus 10 and systems of the present invention. Loading systems such as elevators, conveyors and buckets may be used to transport materials to and from the apparatus 10. These may be of particular use if cycling materials is desired. Atmosphere extraction or vacuuming of the vessels may be achieved by an external device or may be an integral component of the apparatus. Preferably, the transport vehicles may be adapted to extract atmosphere and/or provide power from the vessels 110 such that minimal equipment is required for the apparatus installation. Alternatively, the apparatus 10 may be adapted to be mounted on a vehicle such that sterilisation of food can occur once harvested. This may provide a significant benefit as the quicker sterilisation processes may also trap more pests which can hinder reproduction of said pests and therefore protecting future crops.

In yet a further embodiment, the apparatus 10 may be mounted on a harvester such that harvested foods may be directly deposited within one of the vessels 110 to be deposited in a further storage location after processing. This may also be advantageous as the vacuum in the vessel 110 may assist with drying times of the foods in the vessel 110, which may preserve the foods for a longer period of time. The apparatus 10 may also find use on ships, offshore rigs (such as oil platforms) or other locations as this may allow for a suitable long term food storage option for perishables.

As the sterilisation processes change the atmospheric pressure locally within the vessel 110 and also reduce the oxygen content pests will typically be killed regardless of natural defensive protections or period of lifecycle. Being able to kill pests at unknown stages of a lifecycle is a common industry problem; however with the present apparatus this problem may be remedied. Further, the apparatus 10 adopts sterilisation methods which will typically reduce the required “holding time” of the foods within the vessel 110 compared to known devices or apparatuses. In addition, as chemicals are not used to sterilise the contents of the vessels 110A to 110D (in the case of a four pressure vessel apparatus 10, also see FIG. 6) this also reduces the potential for pests to excrete protective fluids, which also reduces the potential for allergic reactions for persons consuming the foods. Currently, around 70 to 80% of foods are treated with chemicals which may cause allergic reactions, be potentially hazardous to the environment and are expensive to purchase.

The apparatus may allow for sterilisation of organic material when said organic material is being stored, further it may also be advantageous to sterilise the interior of the chamber such that if there are any residual microorganisms within the pressure vessel 110 prior to introduction of the organic material the chamber can also be sterilised in combination with the organic material.

Sterilisation may refer to any process which substantially or completely removes forms of biological material, biological agents, biological pathogens, transmissible agents (such as fungi, bacteria, viruses, spore forms, unicellular eukaryotic organisms such as Plasmodium, and the like) which may be present in a specified region, such as a surface, a volume of fluid, medication, or in the organic material to be stored. Preferably, the sterilisation is performed on a particulate material to be stored in the pressure vessel 110.

There are a number of problems associated with storing a particulate material such as airborne organic matter which may combust during the feeding (loading of organic material) or storing periods. The present apparatus 10 may be adapted to settle airborne materials while in the pressure vessel 110 or being introduced into the pressure vessel 110 by introducing a fluid, such as a liquid or a gas to reduce the settlement time of the particulate material as well as reducing the temperature of the organic material to be stored. Fluid outlets may be provided in the infeed hopper 100 or at a perimeter of the infeed hopper 100 to allow introduction of fluids into the apparatus 10. Further, fluid outlets (not shown) may be provided in each of the pressure vessels 110 such that a sterilisation fluid may be introduced which may also be used to settle airborne particulates within the pressure vessel 110. The pressure vessel 110 fluid outlets (not shown) may also be used to introduce other fluids such as settlement gases to reduce settlement times of airborne particles.

In addition, the pressure vessel 110 may be a double walled vessel, in which a vacuum may exist between the walls such that the pressure vessel 110 is insulated. This may further reduce the potential for excessive external temperatures to combust the material being stored.

Sterilisation methods may include one or more of the following: heat, chemicals, irradiation, high pressure, and filtration. Sterilisation is distinct from disinfection, sanitization, and pasteurization in that sterilisation kills, deactivates, or eliminates forms of life and other biological agents. Sterilisation can be achieved by physical, chemical and physiochemical means. However, with respect to organic materials, such as rice and grain, there are a number of restrictions which render a number of these methods unviable as they will destroy the organic material to be stored or will result in undesirable chemical changes within the organic material to be stored, such as fermentation.

Throughout this specification any reference to ‘food’ or ‘material’ is intended to be a generalised term for organic material or consumable material, but may also include pharmaceutical products, biological products or any other products which may be adversely impacted by exposure to oxygen or microorganisms. While there are numerous references to the apparatus 10 being adapted for food, the apparatus 10 may also be used to store any desired material which may not be identical. The apparatus 10 may also be adapted to store packaged food products, such that the packaged food products can be stored in a relatively sterile environment to prolong the expected life of the food product before potentially being hazardous or undesirable for consumption.

It will be appreciated that the food in the pressure vessel 110 may undergo periodic sterilisation or sterilisation at a selected time to maintain a higher degree of certainty that the contents of the vessel are sterilised or substantially devoid of undesirable microbiological contaminants. For example, if the food is to be stored for four (4) weeks, it may be advantageous to sterilise the vessel and contents contained therein when first deposited in the vessel 110 and at a period two (2) weeks after the first sterilisation. Logs, records and or locks may be provided on an apparatus which records any breach of a seal or loss of sterile field. The internal pressures and gases used (if any) on the materials may be recorded on a batch basis, where a ‘batch’ is the material in a vessel. This may be useful in reducing customs times or delays in foreign jurisdictions as this may allow customs officials to view whether a sterile or safe containment of materials has occurred, which is of particular advantage to jurisdictions with stringent customs, such as Australia.

The apparatus 10 may also have an outfeed similar to the infeed which can be used to direct the stored foods into a receptacle (such as a transport receptacle) or onto the ground when a vessel 110 is to be emptied. The infeed hopper 100 and/or outfeed hopper 120 may alternatively be an auger type device or an air slide. The outfeed hopper 120 and/or the infeed hopper 100 may be formed integrally with the pressure vessel 110. The apparatus 10 may be predominantly formed form at least one of steel, aluminium (aluminum) alloy, Zincalume™, concrete, wood, carbon fibre, brass, or any other desired construction material. Forming a material silo from a metal or metal alloy is generally disadvantageous as this causes internal temperatures to rise and increases the potential for fires and/or fermentation to occur. In one embodiment, the pressure vessel 110 is an elastomeric material which may deform when loaded (i.e. storing food). Preferably, the elastomeric material is resilient such that the elastomeric material will revert to a substantially pre-deformed shape. In one embodiment, an elastomeric material pressure vessel 110 can be depressurised via removal of air (gases) from the pressure vessel 110, which forms a seal between the opposing surfaces of the pressure vessel 110 (in the case of a cylinder, ellipse or ovoid). The seal formed is desirably relatively above the stored food such that the food is between the lower end of the pressure vessel 110 and the seal near to the upper end of the pressure vessel 110.

The apparatus 10 may allow for foods to be contained in a pressurised environment such that the fermentation rate of food being stored can be eliminated or minimised. This is particularly true for anaerobic fermentation. Therefore, pressurising the vessels 110 of the apparatus 10 is a significant advantage. If the apparatus 10 is a multi-pressure vessel apparatus (as seen in FIGS. 1 to 6) each pressure vessel 110 may have a local oxygen content, a local pressure and/or a local humidity, which may collectively be referred to as localised conditions. Each of the localised conditions may be unique for each pressure vessel 110 and may be tailored for the contents of the pressure vessel 110 to maintain a preferred internal environment. This may be advantageous when storing multiple foods at a single time. For example, a two vessel apparatus 10 with a first pressure vessel 110A and a second pressure vessel 110B may have a first set of localised conditions in the first vessel 110A, and a second set of localised conditions in the second vessel 110B, with each vessel 110 optionally storing different contents. It will be appreciated that each vessel 110 may also have the same localised conditions applied thereto.

A multi-pressure vessel apparatus 10 may have an infeed hopper 100 or infeed designed to fill a predetermined number of pressure vessels 110, in which the predetermined number may be one or more vessels 110 at a time. In this way materials diverted into desired vessels 110 to allow separation of stored foods. The hopper 100 may be adapted to allow filling of all vessels 110 at a single time, or may be adapted to fill only selected vessels 110. A feed diverter 104 may be provided to direct flow of materials to the vessels 110.

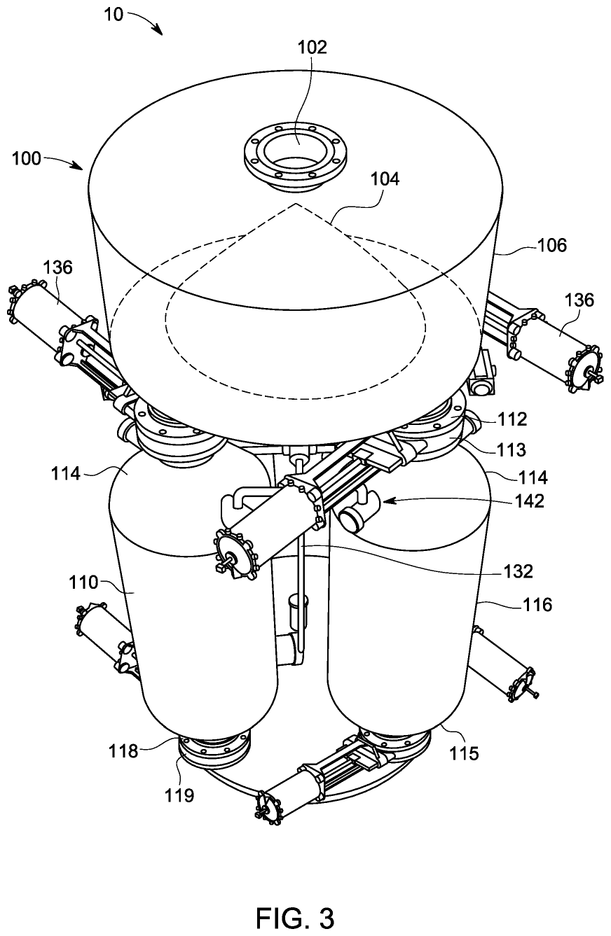

As illustrated in FIGS. 2 and 3, the infeed diverter is a conical shape which diverts the flow of materials into the infeed hopper to at least one infeed 112 of a vessel 110 or adjacent thereto. Vessels 110 may be sealed or otherwise closed such that materials fed into the hopper 100 will be directed to only the open vessels 110. A fluid direction means (not shown) may be used to push, blow, flow or urge foods to an open vessel such that it does not settle on closed vessels 110.

Optionally, the exterior of the apparatus 10 can be used to generate power via photovoltaic cells (not shown) being placed on the exterior of the apparatus 10. This may also have the advantage of providing a further insulation barrier for the apparatus 10, and each respective pressure vessel 110. Wind turbines (not shown) may also be disposed on the exterior of the apparatus 10 to generate power as these apparatuses 10 are typically in locations with high wind loads.

Preferably, an inert gas such as ozone is used in to sterilise water and air, as well as being used to disinfect surfaces. Ozone also has the benefit of being able to oxidize organic matter. Preferably, the ozone is manufactured due to its toxic nature for humans and unstable state at one atmosphere.

Ozone offers many advantages as a sterilant gas; ozone is a very efficient sterilant because of its strong oxidising properties (E=2.076 vs SHE) capable of destroying a wide range of pathogens, including prions without the need for handling hazardous chemicals since the ozone is generated within the steriliser from medical grade oxygen. The high reactivity of ozone means that waste ozone can be destroyed by passing over a simple catalyst that reverts it to oxygen and ensures that the cycle time is relatively short.

In yet another embodiment, ozone is mixed combined with a nitrogen carrier for the sterilisation gas to reduce the volume of ozone input into the apparatus 10.

Another gas which may be suitable for use, for example, may be ethylene oxide (EO,EtO). Ethylene oxide may be used to process items that are sensitive to processing with other methods, such as radiation (gamma, electron beam, X-ray), heat (moist or dry), or other chemicals. Sterilisation may be using ethylene oxide may be carried out between 30° C. and 60° C. with relative humidity above 30% and a gas concentration between 200 and 800 mg/l. It will be appreciated that the temperatures, humidity and concentrations are exemplary only and any predetermined temperatures, humidity and concentrations may be used with the apparatus of the present disclosure. Ethylene oxide is highly effective at sterilising material as it can penetrate porous materials, such as the space between the particulate materials. Ethylene oxide kills a large number of known microorganisms and the sterilisation may be repeated a number of times without impacting the quality of the material in the apparatus 10.

Use of ethylene oxide in a sterilisation method may include a preconditioning phase (in a separate room or cell), a processing phase (more commonly in a vacuum vessel and sometimes in a pressure rated vessel), and an aeration phase (in a separate room or cell) to remove ethylene oxide residues and lower by-products such as ethylene chlorohydrin (EC or ECH) and, of lesser importance, ethylene glycol (EG). An alternative process, known as all-in-one processing, also exists for some products whereby all three phases are performed in the vacuum or pressure rated vessel, which may be employed in at least one embodiment of the system. This latter option can facilitate faster overall processing time and residue dissipation.

The most common ethylene oxide processing method is the gas chamber method. The method uses ethylene oxide, or with other gases used as diluents (chlorofluorocarbons (CFCs), hydro-chlorofluorocarbons (HCFCs), or carbon dioxide).

It is important to adhere to patient and healthcare personnel government specified limits of ethylene oxide residues in and/or on processed products, operator exposure after processing, during storage and handling of ethylene oxide gas cylinders, and environmental emissions produced when using ethylene oxide. As such, the system may be adapted to allow for controlled volumes of gas to be used.

Nitrogen dioxide (NO₂) gas may also be used to assist with sterilisation of the pressure vessel 110 and the material in said pressure vessel 110. NO₂ provides the advantage that it is a rapid and effective sterilant for use against a wide range of microorganisms, including common bacteria, viruses, and spores. The unique physical properties of NO₂ gas allow for sterilant dispersion in an enclosed environment at room temperature and ambient pressure. The mechanism for lethality is the degradation of DNA in the spore core through nitration of the phosphate backbone, which kills the exposed organism as it absorbs NO₂. This degradation occurs at even very low concentrations of the gas. NO₂ has a boiling point of 21° C. at sea level, which results in a relatively high saturated vapour pressure at ambient temperature. Because of this, liquid NO₂ may be used as a convenient source for the sterilant gas. Liquid NO₂ is often referred to by the name of its dimer, dinitrogen tetroxide (N₂O₄). Additionally, the low levels of concentration required, coupled with the high vapour pressure, assures that no condensation occurs on the devices being sterilised. This means that no aeration of the devices is required immediately following the sterilisation cycle. NO₂ is also less corrosive than other sterilant gases, and is compatible with most medical materials and adhesives.

Optionally, other methods of sterilisation which may be suitable include; non-ionising radiation sterilisation methods (such as ultraviolet light irradiation).

With respect to sterilisation for liquids filtration may be used if damaged may be caused by other sterilisation methods, such as storing drug products or vitamins. Filtration sterilised by microfiltration may use membrane filters (not shown). A membrane filter may also be referred to herein as a “microfilter”. The membrane filters can be disposed at the inlet 102 and/or the vessel infeed 112 of the vessel 110. This method of sterilisation may be used for heat labile pharmaceuticals and materials containing protein. A membrane filters with pore size of approximately 0.1 μm to 0.4 μm, but more preferably a pore size of around 0.2 μm, may effectively remove a number of microorganisms. In the processing of biologics, viruses must be removed or inactivated, requiring the use of nanofilters with a smaller pore size of approximately 20 nm to 50 nm. Smaller pore sizes may lower the flow rate, so in order to achieve higher total throughput or to avoid premature blockage, pre-filters might be used to protect small pore membrane filters. It will be appreciated that pressure differentials may also be used to encourage fluid flow into, or out of, the vessels 110.

Membrane filters (not shown) used in production processes are commonly made from materials such as mixed cellulose ester or polyethersulfone (PES). The filtration equipment and the filters themselves may be pre-sterilised disposable units in sealed packaging, or must be sterilised prior to use, generally by autoclaving to avoid damage the filter membranes.

In one embodiment there is provided a multi-pressure vessel apparatus for sterilisation of contents in at least one pressure vessel of the multi-pressure vessel apparatus. The multi-pressure vessel apparatus may comprise two or more pressure vessels 110 in which at least one pressure vessel of the system is adapted to receive particulate material.

Particulate materials may be fed into vessels 110 via gravity fed means, fluid flow, high and low pressure differential systems, or any other conventional means of moving volumes of particulates. Particulates may be any material such as salt, sand, sugar, rice, wheat or any other material in particulate or granular form. Other materials, such as fluids may also be fed into the vessels 110 and stored.

Preferably, the apparatus 10 comprises monitoring means, such as sensors, to detect at least one of; volume of a vessel, remaining volume of a vessel when at least partially filled with contents, the time for filling of a vessel, the weight of a vessel, the percentage of a gas (by volume and/or weight) present inside a vessel, atmospheric pressure in a vessel, blockages, temperatures, fires, leaks, structural damage or any other predetermined sensor to monitor a desired attribute of the apparatus 10.

Optionally, the apparatus 10 has a capturing means (not shown) to record or capture images, video and/or audio. The capturing means may be disposed internally to the apparatus 10, and may be self-contained or separate from the vacuum which can be created in the vessel. For example, a capturing means disposed inside the vessel 110 may have a housing mounted therein which is sealed to retain approximately one atmosphere of pressure within the housing.

If the apparatus 10 is used to process high volumes of food, for example several tonnes of food at a time, the apparatus 10 may have a frame structure (such as that illustrated in FIG. 10) to support the vessels 110. The frame structure may be fabricated from steel or an alloy to retain the structure in a desired location. The frame may also be large enough such that a control room, monitoring room, walkway 170 or gantry may be disposed near to the top of the apparatus 10 such that a person may monitor or inspect the apparatus 10.

In yet another embodiment, the apparatus 10 is a bulk storage apparatus 110 which may be used to store particulate produce in a gas-tight container which enables the stored produce to be subjected to a vacuum. The vacuum may be supplemented by inert gases which suppress or extinguish life, particularly microorganism life.

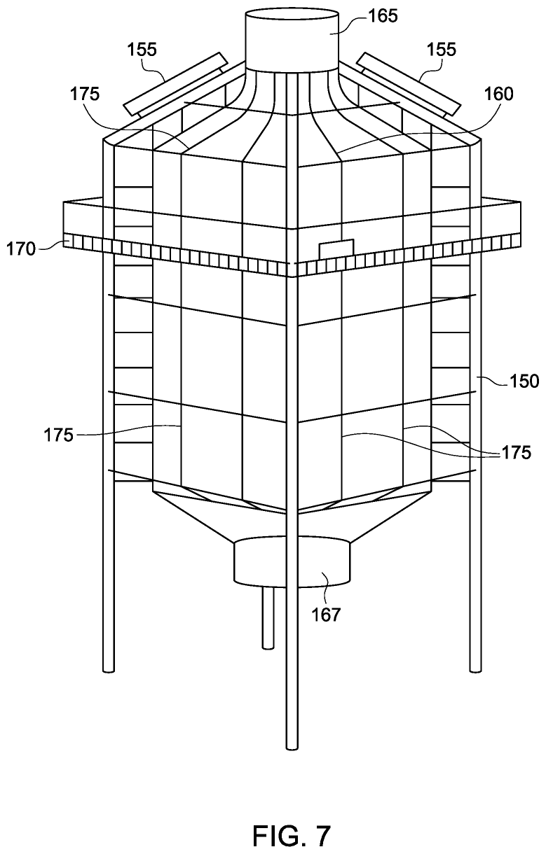

In one embodiment, the storage vessel 110 for the particulate produce may be a flexible vessel 160, or deformable vessel 160, which is preferably impermeable and preferably formed from a resilient material. The vessel 110 is also preferably substantially cylindrical such that the force is evenly distributed around the vessel. However, the vessel 110 may be polygonal without contents as the vessel may be adapted to deform with loading. If the vessel is designed to deform or otherwise change shape when loaded, the vessel may be reinforced with reinforcement means 175 (see FIG. 7) to allow for a generally predetermined deformed vessel shape. The reinforcing means 175 may also be a weave or a braid such that lateral and/or longitudinal deformations may be restricted when the vessel is loaded. The reinforcing means 175 may also be a flexible and/or elastic material such that when the vessel 160 is loaded the vessel 160 may be allowed to deform to accommodate the loaded materials. In one embodiment, the vessel 160 preferably has the means for creating, monitoring and maintaining the partial vacuum through the storage period, and adjusting the vacuum level accordingly to increase or reduce the amount of fluids within the vessel 160.

The deformable vessel 160 may be constructed from polymer, such as polyurethanes and/or rubberized materials, as well as a range of food grade plastic tubing. The vessel maybe reinforced by mesh or ribbing formed from stainless steel, fibre-glass or carbon fibre to increase its tensile strength and to give resistance to external attack from rodents and birds and vandalism. Further, the exterior of the vessel 160 may comprise a puncture resistant or slash resistant material. Preferably, the wall thickness of the deformable vessel is in the range of 5 mm to 500 mm, but more preferably, the wall thickness is between 80 mm to 100 mm. Optionally the wall thickness may be of predetermined varying thicknesses.

The vessel 110, 160 may have a capacity of one tonne or more, and be intended to store the produce subjected to a partial vacuum throughout the storage period with intermittent adjustment of the vacuum level or to subject the produce to a partial vacuum intermittently. Optionally, the atmosphere internal the vessel 160 may be replaced, at least in part, with a life suppressing fluids such as nitrogen, carbon dioxide, ozone or a combination thereof. It will be appreciated that other suppressants may be used, such as chemicals or fluids. An inlet may be used to provide the suppressing fluids or chemicals to assist with sterilisation or preservation of the contents of the vessel. In addition, desiccating and oxygen absorbing agents may be added to the bag in ways that do not contaminate the produce, however this is optional.

Additional ancillary equipment may be used with the apparatus 10 to introduce controlled atmosphere gases or to evacuate the atmosphere in the deformable vessel. Power for the apparatus or ancillary equipment therefor may be provided at least in part via wind turbines or photovoltaic cells.

The pressure of the vacuum might be adjusted according to the percentage (by volume) of gasses mentioned above when these are present. The axial cross section of the vessel 110, 160 is preferably circular, ovoid or elliptical, but may be any other predetermined shape. The deformable vessel may have conical ends top and bottom which are supported by a structural support frame. The deformable vessel 160 preferably rests on radial arms that meet at, or near to, a central axis of the deformable vessel, which gives access to the hatch. The lower portion of the vessel 160 may be attached to the deformable vessel 160 at an outlet 167. The outlet may be an outlet collar 167 type structure which is generally rigid to provide a known output rate.

In an exemplary embodiment, the frame 150 for the deformable vessel 160 is at least the same height, but preferably larger, than the deformable vessel 160 such that the vessel can be suspended relatively above the ground. Suspending the vessel above the ground provides a number of advantages, particularly allowing a transport vehicle to be positioned below the outlet of the vessel such that the outlet may be opened to fill the vehicles receptacle. The frame 150 comprises a supporting structure with a plurality of radial arms which are used to support the vessel 110, 160 near to the top of the vessel and preferably near to the bottom and/or near to the centre of the vessel. The radial arms may be secured to the vessel at predetermined anchor points or may be secured via an attachment means, for example bolts or friction plates. The radial arms may also be used to assist with loading of the vessel or may be used to assist with a desired deformation of the vessel. In one example, the radial arms may impart a circular shape to the vessel such that pressure may be more evenly distributed. It will be appreciated that the deformable vessel may deform radially inwardly relative to a vertical central axis of the vessel when a vacuum is imparted to the vessel.

In yet a further embodiment, the vessel 110 comprises an upper conical shape 114 which follows the contour of the upper radial arms. This may assist with ensuring that the material poured through the inlet piles in a conical shape at the bottom 115 of the vessel and inclining upwards radially from the upper extremity of the cylindrical wall 116 supported by the supports near to the underside of the lid to minimize the volume of empty space in the vessel and therefore minimize the power required to maintain a vacuum in the vessel 110, 160. Optionally, the upper portion of the frame 150 comprises photovoltaic cells or power generation devices 155 which can supply power to a battery or electronics of the apparatus 10, such as the motor for the vacuum pump 130. The motor for the vacuum pump may be operated by a control box, which receives signals from, one or more sensors which measure or otherwise detect the pressure (vacuum level) in the vessel. The control box has a communication means adapted to communicate with a network, such that data can be issue to a computer or portable device, such as a smart phone or notebook. Data issued to a computer or device may be via an application or via the internet, such that only predetermined users may view the data. The data may relate to process information, potential processing issues, errors, anticipated rates of production, images, videos or audio from capture devices, or any other data sets or information.

A vacuum outlet may be connected to one or more vessels 110, 160 of the apparatus 10 comprising activated carbon which may absorb carbon dioxide form the vessel 110, 160. Other absorbing means or capture means may be used for the gases from the vessel, if any are present. The vacuum pump may also be adapted to aerate produce if desired. The apparatus 10 may be fitted with warning devices which may be used to issue alerts to persons monitoring the device.

The vessel may be split into distinct sections, comprising a cylindrical midsection 116 and conical top portion 114 and a conical bottom portion 115. The conical top portion preferably comprises a sealable hatch or opening which allows loading of materials into the vessel. The bottom portion of the vessel is preferably adapted to allow for extraction or unloading of foods stored or passed through a vessel.

The initial removal of vessel atmosphere of the deformable vessel 110 may be achieved by a mobile power unit with a suitable pump, with an installed ancillary pump and power unit to maintain the vacuum within the sealed vessel. Alternatively, the ancillary equipment may be used both to create the initial vacuum in a vessel 110 and maintain it. The ancillary equipment may be powered by solar power, for example, photovoltaic cells 155 with or without battery backup, mains power or any other power source which may sustain power for the vessel size. The vessel 110 may have an outlet for gas-removal, either at the top, the side or at the bottom. The included ancillary equipment may include a vacuum pump (or other means for extracting gas), solar panels with inverter and back-up batteries supplied for the vessel 110.

The level of vacuum needed to sterilise the produce may be modified by the level of life suppressing or life denying gases optionally injected into the vessel. An optimum pressure to gas ratio may be specific for the foods being stored, in which the optimum pressure to gas ratio may relate to a minimum dwell time for the food or may be related to a minimum sterilisation cost.

A frame supporting structure 150 may be adapted to support either a deformable vessel or rigid vessel. Referring to FIG. 6, this structure is shown as a frame 150 from which a deformable vessel 160 is suspended. In the case of rigid vessels, these may be suspended, braced, retained or stood. The frame 150 may comprise a ring of braced vertical studs with radial struts top and bottom to support a vessel, along with ties to the sides of the vessel. The top of the vessel 160 may converge toward a solid ring or collar 165 supported by the struts. The collar 165 may also provide a means for access to the vessel interior. Likewise the bottom of the vessel 160 may rest in a conical housing supported by the said bottom struts, and housing a lower collar 167 and access hatch.

The frame 150 may also be able to carry the ancillary equipment on a working platform and with, ladders to provide access to the top hatch, and for access from the ground.

While the present disclosure is made with reference to a fixed installation, for example, an onsite apparatus 10, the apparatus may be adapted for other purposes and/or settings, such as a vehicle plane or boat. If the vessel 110 is a deformable vessel 110, it may also be adapted to be a sleeve or lining inside an existing silo, or in a shipping container. Retrofitting to existing silos or machines may provide a significant value to existing business and installations. This may provide a number of advantages for retrofitting existing devices to reduce overall equipment costs while also taking advantage of the present disclosure. Further, this may be of particular advantage for importations of organic material such as wood, animal products and foods which may require to be quarantined or move through customs at ports and airports. The apparatus 10 may also be used to store harvest direct from combine harvesters, and used to aerate and dry the grain immediately on harvesting, to protect it in moist conditions, and to prevent even immediate problems from arising, such as sprouting or moulds. Likewise road or rail or sea transport could be done under the optimal conditions supplied by the invention. Other applications may also be available for the present disclosure and the above uses are exemplary only and are not limiting.

Inert gasses may include noble gases and gasses which are inert under particular conditions in predetermined systems such that unwanted chemical reactions substantially do not take place under the predetermined conditions. For example, the use of O₃ (ozone) under a predetermined pressure may be considered to be an inert gas.

Preferably the pressure vessels are gravity fed such that particulate materials can be deposited in the vessel to fill a larger volume more quickly than conventional sterilisation chambers which are typically disposed horizontally.

This disclosure may provide for to a food sterilising apparatus capable of successively carrying out steps of sterilising and cooling food contained in rigid containers.

In yet another embodiment, the vessel 110 may instead be a processing room (not shown) in which foods may be deposited. The processing room may be adapted to be pressurised, and may have an infeed for injecting a mixture of ozone gas and an inert gas or gases. The food may be sterilised by the pressurisation (vacuum) of the room. Further sterilisation processes may be employed before or after the pressurisation of the processing room, for example sterilisation gases or chemicals may be introduced into the vessel. A gas control device can be used to regulate the gases injected into the processing room such that a mixing ratio of gases and/or pressures may be obtained.

In yet a further embodiment, the apparatus 10 is fitted with a plurality of feed pipes are connected with a fluid mixing means. The feed pipes typically allow the flow of gas into the mixing means, but may also allow for liquids to flow into the mixing means. The fluid mixing means may allow for fluids and/or gases to be mixed, while not undergoing chemical reactions. This may allow for two or more distinct fluids to be present in the fluid mixing means to be used within a vessel 110. An injection device is provided to be in communication with the vessel 110 such that fluids may be injected into the vessel 110 which may assist with sterilisation, wetting, or causing a desired reaction or deactivation. A mixing controller is adapted to maintain a predetermined mixing ratio of fluids within the fluid mixing means, such as carbon dioxide gas and nitrogen gas. Gas density sensors may also be provided to ensure that mixing ratios are within predetermined threshold limits. Electromagnetic valves can be used with the fluid mixing device to reduce the potential for combustion of fluids.

The gas density sensors can be adapted to sense the density of fluids such as; ozone gas, carbon dioxide gas, nitrogen gas or any other predetermined fluid. The sensed densities can be communicated to a control panel, computer or display for a user such that the user may monitor the vessels 110 of the apparatus 10. The electromagnetic valves are preferably provided for the feed pipes to open and close the feed pipes.

To sterilise food in the apparatus 10, the food is firstly deposited in a vessel. The vessel is then sealed in a gas-tight manner such that the internal atmosphere of the vessel may be altered artificially. The vacuum pump 130 may then be used to extract at least a portion of the atmosphere within the vessel 110. Once the atmosphere in the vessel 110 is at a desired pressure level, the pressure is maintained for a period of time, or ‘dwell time’. After the dwell time, a gas may be introduced into the vessel 110 to further raise the certainty of sterilisation of the food contents. After the gases have been within the vessel 110 for a desired period of time, the gases may be extracted, replaced or vented. The food may then be stored in the vessel 110 until packaging or transport.

The mixing ratio of ozone gas and an inert gas or gases should be set as respective appropriate values according to the kind of food, and in case of some kinds of food, it is possible to use separately carbon dioxide gas or nitrogen gas. Mixing gases may have a number of benefits, for example when ozone gas is used with an inert gas or gases in the process, a synergetic effect may be achieved to both effect sterilization and deoxidization of the inert gas or gases can be obtained. For example, if carbon dioxide gas is used as the inert gas, it may sterilise the inside of the food, when ozone gas sterilises the surface of the food at the same time. Also, if nitrogen gas is used as an inert gas, it prevents deterioration of the food due to the excessive oxidation of ozone gas, and it prevent the food from changing colour and from emitting offensive odours.

Optionally, a plurality of pressure vessels 110 may be used in conjunction with a grain elevator. A grain elevator may be used typically with two to twenty silos or storage units, which can be replaced by the pressure vessels of the apparatus 10. The grain elevator may include a bucket elevator or a pneumatic conveyor which may transport grain or other food material to the infeed of a pressure vessel to be stored for a period of time.

In yet another embodiment, the vessel may optionally comprise sensors to detect persons in the vessel 110. Preferably, the vessel is not accessible for persons, however using industrial equipment there may be the potential for accidents. As the vessel 110 will typically be an enclosed space with a lack of oxygen, if the vessel 110 detects a person within the vessel, the apparatus may be adapted to inject oxygen into the vessel to maintain breathable levels of atmosphere for the person inside the vessel until they can be safely removed from the vessel 110.

The apparatus 10 may expand and contract in vertical height based on exposure to heat, such as the sun and the internal temperature fluctuation of the material stored in a vessel 110. As such, the apparatus 10 may be fitted with thermal expansion means or expansion joint; such that the vertical height of the apparatus, or part thereof, may change based on the temperature of the vessels and the coefficient(s) of thermal expansion of the apparatus 10. If there is a frame supporting the apparatus 10, the frame may also be adapted to expand and contract with the apparatus 10.

In yet another embodiment, the apparatus 10 may be adapted to allow for cycling of materials between a first pressure vessel 110A and a second pressure vessel 110B. Cycling may be achieved by at least partially extracting the material contained in a first pressure vessel 110A and moving the material to a second pressure vessel 110B. Moving the material may expose the material to external environments or non-sterile environments for a period of time, and as such it is preferred that at least one sterilisation process is carried out to ensure the material has again undergone sterilisation and/or is placed in a sterile field.

A safety release valve may be disposed on the apparatus which is adapted to rapidly expel internal gases in vessels and/or rapidly alter the atmospheric conditions internal to the vessel 110. It will be appreciated that these safety release means may be disposed near to the top of the vessel 110 to avoid injuries to persons nearby in the event of activation. A wetting or fire suppression system may also be disposed within at least one vessel and/or on the exterior of the apparatus 10.

In yet a further embodiment, the internal temperatures of the vessels 110 can be manipulated or cycled between vessels. This is a significant advantage over the prior art as this may further provide for the use of metal units or pressure vessels 110. As metal materials may heat up and store heat which is then likely transferred to the material within the vessels 110, manipulation of oxygen and or temperature will reduce the potential for explosions or fires which are risks with known storage devices. As such, known units will generally use concrete or masonry silos, which are porous and therefore cannot be pressurised like the present apparatus 10.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. An apparatus for sterilising material, the apparatus comprising;
a pressure vessel;
the pressure vessel comprising a vessel infeed and a vessel outlet;
the vessel infeed and vessel outlet are adapted to form respective seals with the pressure vessel such that a desired pressure can be maintained in the pressure vessel; and
wherein the pressure vessel is depressurised such that the material deposited in the pressure vessel is sterilised and at least a portion of the inner surfaces of the pressure vessel are sterilised, such that the material deposited into the pressure vessel can be stored therein for a predetermined period of time.

2. The apparatus as claimed in claim 1, wherein the pressure vessel is a plurality of pressure vessels, in which at least a first pressure vessel and a second pressure vessel of the plurality of pressure vessels are in communication with each other.

3. The apparatus as claimed in claim 2, wherein when the first pressure vessel is depressurised, the second pressure vessel is partially depressurised by opening a fluid conduit between the first pressure vessel and the second pressure vessel.

4. The apparatus as claimed in claim 2, wherein the material deposited into the first pressure vessel can be cycled to the second pressure vessel to be stored.

5. The apparatus as claimed in claim 1, wherein a sterilant gas is injected into the pressure vessel at a predetermined pressure.

6. The apparatus as claimed in claim 5, wherein the gas is an inert gas at a predetermined pressure.

7. The apparatus as claimed in claim 6, wherein the desired pressure and the predetermined pressure are the same pressure.

8. The apparatus as claimed in claim 1, wherein an infeed hopper is disposed above the pressure vessel and adapted to direct material to the inlet of the pressure vessel.

9. The apparatus as claimed in claim 1, wherein an outlet hopper is disposed relatively below the pressure vessel and adapted to receive material from the outlet of the pressure vessel.

10. The apparatus as claimed in claim 1, wherein the pressure vessel is a vertical pressure vessel such that material is gravity fed into the pressure vessel.

11. The apparatus as claimed in claim 1, wherein the pressure in the vessel is adapted to cycle between one atmosphere to the desired pressure.

12. The apparatus as claimed in claim 11, wherein the pressure vessel is cycled three times between one atmosphere to the desired pressure.

13. The apparatus as claimed in claim 12, wherein the three cycles occurs during a period in the range of 15 minutes to 45 minutes.

14. A method of sterilising material in a multi-pressure vessel apparatus, the method comprising the steps of;
depositing a material in a first pressure vessel;
sealing the first pressure vessel and creating a depressurised atmosphere in the first pressure vessel;
maintaining the depressurised atmosphere in the pressure vessel for a first predetermined holding time;
depositing a material into a second pressure vessel;
sealing the second pressure vessel and opening a fluid conduit between the first pressure vessel and the second pressure vessel such that the depressurised atmosphere from the first pressure vessel is used to partially depressurise the atmosphere of the second pressure vessel;
closing the fluid conduit between the first pressure vessel and the second pressure vessel; and
creating the depressurised atmosphere in the second vessel for a second predetermined holding time.

15. The method of claim 14, further comprising the step of injecting a gas into the first pressure vessel and/or the second pressure vessel during the respective holding time.

16. The method of claim 14, further comprising the step of opening the seal of the first pressure vessel such that material deposited in the first pressure vessel can be extracted via gravity feed.

17. The method of claim 14, further comprising the step of cycling the pressure in at least one of the first vessel and the second vessel from between one atmosphere and the depressurised atmosphere.

18. The method of claim 14, further comprising the step of increasing the pressure in the first pressure vessel prior to the step of creating a depressurised atmosphere in the first pressure vessel.
